(19) United States
(12) Patent Application Publication
(10) Pub. No.: US 2010/0137919 A1
(43) Pub. Date: Jun. 3, 2010

(54) BONE SCREW

(76) Inventor: Dietmar Wolter, Hoisdorf (DE)

Correspondence Address: MORRISON & FOERSTER LLP, 1650 TYSONS BOULEVARD, SUITE 400, MCLEAN, VA, 22102, US

(21) Appl. No.: 12/520,744

(22) PCT Filed: Dec. 12, 2007
(86) PCT No.: PCT/EP2007/010896

§ 371 Date: Jan. 22, 2010

(30) Foreign Application Priority Data
Dec. 20, 2006 (DE) 10 2006 060 933.6

(51) Int. Cl. A61B 17/86

(52) U.S. Cl. 606/308

(57) ABSTRACT

The invention relates to a bone screw with a shaft that defines a longitudinal axis and with a head that is configured as a thickened portion, said shaft and head being threaded. The thread of the shaft (bone thread) merges in an uninterrupted manner into the thread on the flared area of the head adjoining the shaft. As a result, part of the threaded head lies in the osseous tissue, giving a larger contact surface between bone screw and bone and improving the distribution and transmission of the loads and forces.

### REFERENCE TO RELATED APPLICATIONS

This application is a national stage application under 35 USC 371 of International Application No. PCT/EP2007/010896, filed Dec. 12, 2007, which claims the priority of German Patent Application No. 10 2006 060 933.6, filed Dec. 20, 2006, the contents of which prior applications are incorporated herein by reference.

### FIELD OF THE INVENTION

The invention relates to a bone screw with a shaft that defines a longitudinal axis and with a head that is configured as a thickened portion, said shaft and head being threaded, and to a fixation system for bones that comprises a connecting support and a bone screw.

### BACKGROUND OF THE INVENTION

Bone screws are used in fixation systems for bones with a connecting support in order to connect bone fragments to each other.

The connecting support can be a bone plate, in which case the screw head engaging in a hole of the bone plate is usually blocked at a stable angle with respect to the plate. A connection between bone screw and bone plate with angle stability leads to a gain in stability of the overall assembly. When the screw head is blocked in the hole in the plate, the transmission of forces and loads takes place along the full length of the thread lying in the bone (Wolter et al., 1999, Universeller Titanfixateur interne—Entwicklungs-geschichte, Prinzip, Mechanik, Implantat-gestaltung und operativer Einsatz, Trauma und Berufskrankheit, vol. 1, 307-319). The acting loads and forces are distributed across the bone in proportion to the contact surface of the bone screw. The effectiveness of the system, i.e. the optimal distribution of the loads and forces over a large area, therefore depends on the contact surface between bone screw and bone. Tests have shown that the longest screws are the most effective. However, long screws cannot always be used since, for example, limits are imposed on the screw length by the anatomy of the bones.

From DE 43 43 117 A1, it is known that the angle stability is achieved by material deformation in the area of the contact surfaces of the screw head and the wall of the hole in the plate.

During the deformation of the material of the hole wall, which can be designed as a material lip according to DE 198 58 889 A1 for example, formation of a chip is possible, and this chip may emerge from the entry plane of the hole in the plate. This is also seen as a disadvantage for reasons relating to compatibility, not least because the chips represent a foreign body that can trigger corresponding foreign-body reactions in the surrounding tissue. Moreover, there is a risk of emerging chips irritating the adjacent tissue.

In bone screws of the type mentioned at the outset, the thread of the screw head usually differs from that of the shaft. For example, the screw head of the screw disclosed in DE 198 58 889 A1 is blockable at a stable angle and has an independent thread with a different thread geometry than that of the screw shaft. The two threads have to meet different functional requirements, which necessitates different designs.

The thread in the area of the shaft, which is normally referred to as the bone thread, serves for connection to the bone. The bone thread is configured such that a greater spacing is provided between the individual thread flanks and, at the same time, the thickness of the root of the thread is reduced. In this way, the ratio between the amount of bone substance and the amount of implant material is shifted in favor of the amount of bone substance in the contact area. Since the implant material (e.g. steel/titanium), i.e. the bone screw, has substantially greater strength than the bone, this abovementioned ratio is favorable for the transmission of forces and loads, without causing destruction of the bone in the customary load range.

Other conditions exist in the area of the connection between the screw head and the plates. Comparatively solid materials are in contact here, e.g. pure titanium (plate) in contact with a titanium alloy (screw head). The fact that pure titanium has soft material properties and that a titanium alloy has harder material properties leads to a deformation of the material in the area of the wall of the hole in the plate if a threaded conical screw head made of a titanium alloy is screwed into the hole made of pure titanium. In contrast to the bone thread of the screw, the thread on the screw head is produced here in such a way that the thread flanks have smaller spacings and shallower thread depths. When a connection with angle stability is obtained between bone plate and screw, surface transmission of loads takes place in the contact area between implant and bone.

In previous connections with angle stability between plates and bone screws, the bone thread ends in the neck area of the screw. The neck area itself is often thicker than the core diameter of the screw and is not itself threaded. Tests have shown that the unthreaded neck area is a particularly critical area of the bone screws. In this area, fatigue fractures can occur under excessive loads.

### SUMMARY OF THE INVENTION

The object of the invention is to make available a bone screw that avoids the aforementioned disadvantages to the greatest possible extent.

According to claim 1, the bone thread of the shaft merges in an uninterrupted manner into the thread of the flared area of the head of the bone screw. In other words, the screw according to the invention does not have an actual neck area.

The invention has recognized that, by continuing the thread in an uninterrupted manner from the shaft into the flared area of the head, a greater contact surface is created between bone screw and bone, resulting in improved distribution and transmission of the loads and forces across the greater load transmission area.

Surprisingly, it has also been found that engagement of the screw head in the osseous tissue via its flared area directly adjoining the shaft is not a disadvantage as was once assumed (DE 43 43 117). Instead, a larger contact surface between bone screw and bone is additionally created by part of the flared area of the screw head passing through the through-hole in the plate and into the osseous tissue. The loads and forces can thus be transmitted with better distribution.

Advantageously, the thread on the flared area of the head of the bone screw has the thread pitch of the bone thread. The thread depth of the thread on the flared area is expediently shallower than the thread depth of the bone thread.

In order to ensure an effective blocking of the screw head in the wall of the hole in a bone plate, the head area, in a preferred embodiment of the invention, has a multi-run thread, for example a double-run thread, with the thread pitch being maintained. As the bone screw is screwed into the bone, the lower head area engaging in the bone with its double-run thread can utilize the thread already formed in the bone by the bone thread of the shaft. This is because the bone thread of the screw has cut a thread into the wall of the bone hole, such that a flank of the double-run thread of the head here has direct contact with the bone tissue. The second thread turn of the double-run thread, however, has to cut independently into the bone. To be able to do this efficiently, it is advantageous that the thread edges of the double-run thread are self-tapping. The thread of the head engaging in the bone can expediently have elongate recesses so as to be able, if appropriate, to take up any bone substance that is formed during the cutting process.

According to a preferred embodiment of the invention, the head area adjacent to the shaft widens, relative to the longitudinal axis of the shaft, at an angle of 10 to 28 degrees, preferably of 12 to 25 degrees, more preferably of 13.5 to 15.5 degrees, particularly preferably of 14.5 degrees, and it is preferably a flat head or cone, a fillister head, a spherical head or a pear-shaped head.

The invention has recognized that within the aforementioned angle ranges, particularly within the preferred angle ranges, the undesired formation of chips mentioned in the introduction can be avoided to a very large extent. This is confirmed by Examples 2 to 5. It is assumed that the elongate head shape of the bone screw leads to more uniform and stronger pressing of the deformed material in the wall of the hole and, in this way, chips that may emerge from the hole can be effectively avoided.

A further advantage of the head shape predetermined by the preferred angle ranges is the improved arrangement of a tool engagement part on the screw head, for example a torx socket or hexagon socket for receiving a suitable screwdriver tool. Since the screw head narrows only slowly in the direction of the shaft, it is possible to make the recess for the tool, for example for a screwdriver, deeper and, therefore, of smaller diameter, without weakening the wall in the head area. If the head of the bone screw is a cone, then the angle of the cone of the recess expediently corresponds to the angle of the head cone. Here, the wall thickness between the recess and the outer contour of the head remains unchanged. A trumpet-shaped configuration of the recess is also advantageous in which the entry point of the tool engagement part is wider and this width decreases in a trumpet shape toward the interior of the head. This ensures that the force transmission is generated over a larger contact surface and, consequently, that destruction of the recess in the screw head and also in the area of the tool, e.g. the blade of the screw driver, is avoided.

The invention further relates to a fixation system for bones with a connecting support having at least one through-hole into which the bone screw according to the invention is fitted, the connection between bone screw and connecting support having angle stability, preferably multidirectional angle stability.

### BRIEF DESCRIPTION OF THE DRAWINGS

Further details and advantages of the invention will become clear from the following description of the attached drawings of preferred embodiments and from the examples. In the drawings:

FIG. 1 shows a front view of a bone screw according to the invention;

FIG. 2 shows a cross section through a bone screw according to the invention;

FIG. 3 shows a cross section through a bone screw according to the invention arranged in a bone and blocked in a hole of a bone plate;

FIG. 4 shows a top view of a screw head with a hexagon socket (a) or torx recess (b);

FIG. 5 shows a bone screw screwed into a hole with hole lip;

FIG. 6 shows the hole according to FIG. 5 after removal of the bone screw;

FIG. 7 shows another bone screw screwed into a hole with hole lip;

FIG. 8 shows the head part of the bone screw according to FIG. 6 after removal from the hole; and

FIG. 9 shows the hole according to FIG. 7 after removal of the bone screw; and

FIG. 10 shows a drill device.

### DESCRIPTION OF THE INVENTION

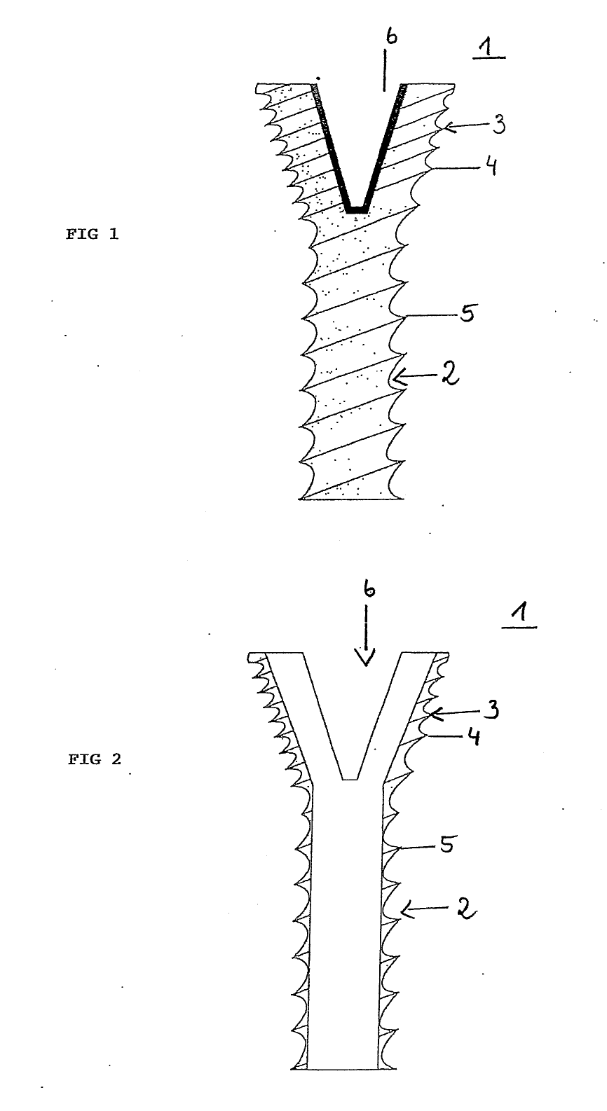

FIGS. 1 and 2 show a bone screw 1 according to the invention in a front view and in cross section, respectively. The bone screw 1 has a shaft 2 with a bone thread 5 and has a cone-shaped head 3 with a thread 4. The bone thread 5 merges in an uninterrupted manner into the multi-run (double-run) thread 4 on the flared area of the head 3, the thread depth of the thread 4 being shallower than that of the bone thread 5. The thread 4 has the same thread pitch as the bone thread 5. Arranged on the top face of the head 3 there is a tool engagement part 6 which is in the form of a cone-shaped recess into which a corresponding tool can engage.

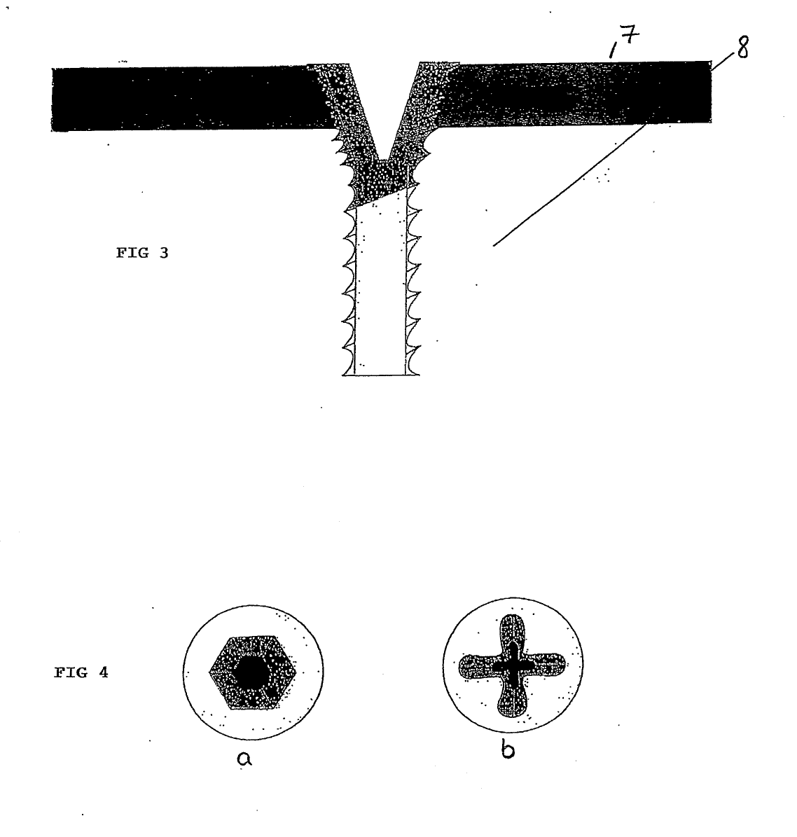

FIG. 3 shows the bone screw 1 from FIGS. 1 and 2 screwed into a bone 8 and blocked with its head 3 in a hole of a bone plate 7. The material of the hole deformed by the head 3 has pressed into the wall of the hole and has. compacted the material at these locations. The lower head part 3 flared in a cone shape and directly adjoining the shaft engages in the osseous tissue 8.

The bone screw can be screwed in and removed via a tool engagement part which is shown in FIG. 4 and which is in the form of a recess for a hexagon key (a) or torx (b).

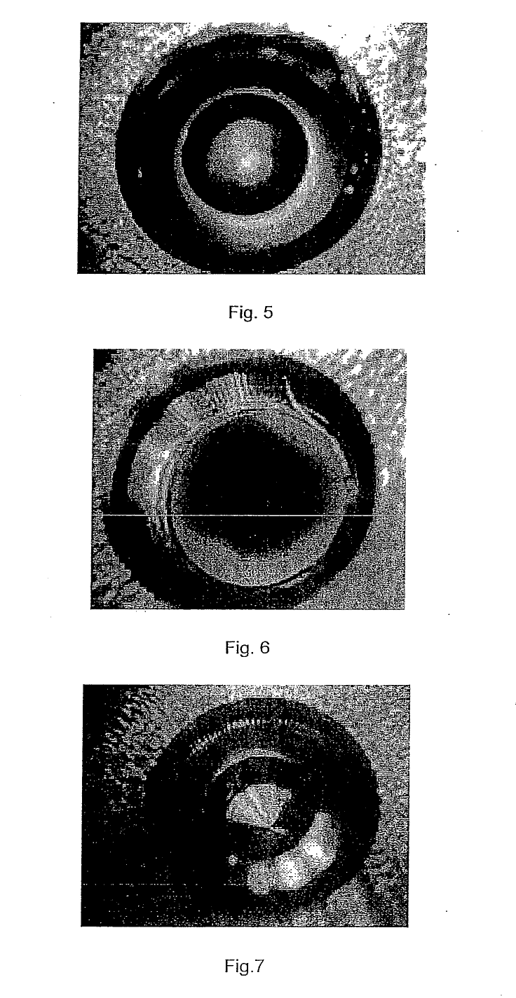

FIG. 5 shows a bone screw 1 according to FIGS. 1 to 3 which is blocked in a plate hole with lip. A chip measuring ca. 5 mm in length rises from the plate hole.

FIG. 6 corresponds to FIG. 5 and shows the plate hole after removal of the bone screw 1. The lip is furrowed. A chip measuring ca. 5 mm in length is shorn off.

FIG. 7 corresponds to FIG. 5. A bone screw according to the invention with a 14.5° conical head has been used. There is no chip rising from the plate hole.

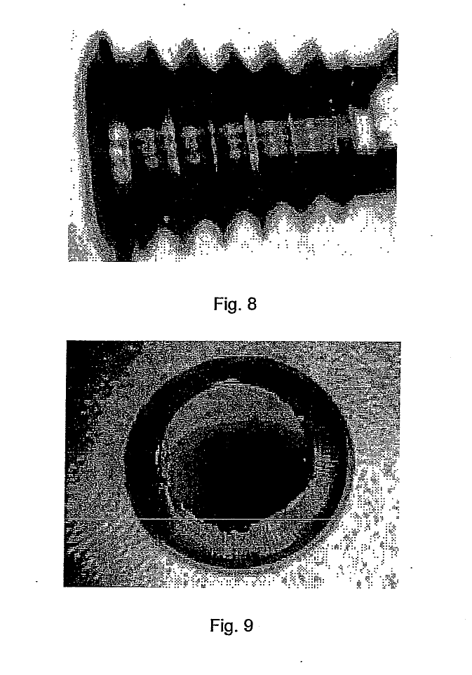

FIG. 8 shows the head part of the 14.5° conical head bone screw from FIG. 7 after removal from the plate hole.

FIG. 9 corresponds to FIG. 7 and shows the hole in the bone plate after removal of the bone screw. No chip formation is visible. The lip is only slightly furrowed.

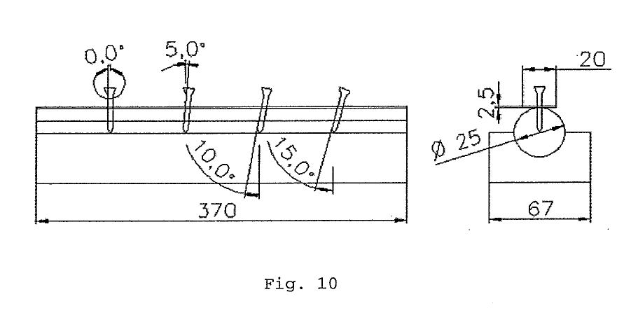

FIG. 10 shows the screwing-in tests performed in a drill device. The left-hand side of the figure shows the drill device with the flexible slide jig that permits drilling at the various screwing-in angles into the bone. The right-hand side shows how the tifix® MINI 1 screw is screwed into the synthetic bone and blocked in the lip.

To assess the chip formation during screwing-in of the bone screw according to the invention as a function of the angle of the flared area of the head, the tests described in the following examples were performed.

### EXAMPLE 1

### Test Setup

Standard plates of titanium 1 measuring 125×20×2.5 (L×B×H) with 12 holes. Screwing of bone screws (type tifix® MINI 1—titanium 4 (high-strength pure titanium)) into synthetic bone, with different conical head geometry (14.5°, 16°, 17.5°) and defined screwing-in angles (5°, 10°). Starting torque=2.5-3 Nm.

### EXAMPLE 2

Bone screws with a 17.5° conical head were tested at a screwing-in angle of 5° according to the test setup described in Example 1. In 3 of 5 cases, chip formation was visible in the blocked state.

### EXAMPLE 3

Bone screws with a 16° conical head were tested at a screwing-in angle of 5° according to the test setup described in Example 1. In 3 of 5 cases, chip formation was visible in the blocked state.

### EXAMPLE 4

Bone screws with a 16° conical head were tested at a screwing-in angle of 10° according to the test setup described in Example 1. In 1 of 5 cases, chip formation was visible in the blocked state.

### EXAMPLE 5

Bone screws with a 14.5° conical head were tested at a screwing-in angle of 5° according to the test setup described in Example 1. In no case was chip formation visible in the blocked state.

The test results from Examples 2 to 5 show that, as the cone angle of the head of the bone screw decreases, fewer chips emerge from the hole in the bone. No chips are generated at a cone angle of 14.5°, since significantly lower forces appear to be generated by this geometry. This has the effect that material is not shorn off from the screw head or from the screw hole, and the lip too suffers much less damage.

1. A bone screw comprising:
a shaft defining a longitudinal axis and comprising a bone thread; and
a head is configured as a thickened portion and comprising a flared area with a thread merging in an uninterrupted manner into the shaft's bone thread,
wherein when a bone plate is fixed to a bone by means of the bone screw, part of the flared area of the head of the bone screw passes through a through-hole in the bone plate and comes to lie in osseous tissue.

2. The bone screw of claim 1, wherein the thread on the flared area of the head has a substantially identical thread pitch as that of the shaft's bone thread.

3. The bone screw of claim 1 or 2, wherein the flared area of the head has a multi-run thread.

4. The bone screw of claim 1, wherein the thread on the flared area of the head is a self-tapping thread.

5. The bone screw of claim 1, wherein a thread depth of the thread on the flared area of the head is shallower than a thread depth of the bone thread on the shaft.

6. The bone screw of claim 1, wherein the flared area of the head widens, relative to the longitudinal axis of the shaft, at an angle of 10 to 28 degrees.

7. The bone screw of claim 1, wherein the head extending from the shaft is a flat head or cone, a fillister head, a spherical head or a pear-shaped head.

8. The bone screw of claim 1, wherein the head comprises a tool engagement part.

9. The bone screw of claim 1, wherein the flared area of the head lying in the osseous tissue comprises chip-receiving areas.

10. A fixation system for bones with a connecting support having at least one through-hole into which a bone screw according to claim 1 is fitted, the connection between bone screw and connecting support having angle stability.

11. The fixation system of claim 10, having multidirectional angle stability.

12. The bone screw of claim 3, wherein the multi-run thread is a double-run thread.

13. The bone screw of claim 6, wherein the flared area of the head widens, relative to the longitudinal axis of the shaft, at an angle of 12 to 25 degrees.

14. The bone screw of claim 6, wherein the flared area of the head widens, relative to the longitudinal axis of the shaft, at an angle of 13.5 to 15.5 degrees.

15. The bone screw of claim 6, wherein the flared area of the head widens, relative to the longitudinal axis of the shaft, at an angle of 14.5 degrees.
